SEQUENCE LISTING (4 sequences)

SEQ ID NO: 1 - protein, 192 aa, Felis catus
MGSCECPALLLLLSLLLLPLGLPVLGAPPRLICDSRVLERYILEAREAENVTMGCAEGCSFSENITVPDTKVNFYTWKRMDVGQQAVEVWQGLALLSEAILRGQALLANSSQPSETLQLHVDKAVSSLRSLTSLLRALGAQKEATSLPEATSAAPLRTFTVDTLCKLFRIYSNFLRGKLTLYTGEACRRGDR

SEQ ID NO: 2 - DNA, 579 bp, Felis catus
atgggcagctgcgagtgtcctgctctcctgctgctgctgagtttgctgctgctgcctctgggcctgcctgtgctgggagcacctcctagactgatctgcgacagccgggtgctggaacggtacatcctggaagcccgcgaggccgagaatgtgaccatgggatgtgccgagggctgcagcttcagcgagaacatcaccgtgcccgacaccaaagtgaacttctacacctggaagagaatggacgtgggccagcaggccgtggaagtgtggcagggactggccctgctgtctgaggccatcctgagaggacaggctctgctggccaacagcagccagcctagcgaaaccctgcagctgcacgtggacaaggccgtgtcctccctgagaagcctgaccagcctgctgagagcactgggagcccagaaagaggccacctctctgcctgaggccacatctgccgcccctctgagaaccttcaccgtggacaccctgtgcaagctgttccggatctacagcaacttcctgcggggcaagctgaccctgtacaccggcgaggcttgtcggagaggcgacagatga

SEQ ID NO: 3 - DNA, 2928 bp, Artificial Sequence
  transgene cassette
  5′ ITR (1)..(130)
  CB promoter (582)..(863)
  Chimeric intron (958)..(1930)
  CDS FelEpo (1942)..(2520)
  rBG poly A (2584)..(2710)
  3′ ITR (2799)..(2928)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggcagctgcgagtgtcctgctctcctgctgctgctgagtttgctgctgctgcctctgggcctgcctgtgctgggagcacctcctagactgatctgcgacagccgggtgctggaacggtacatcctggaagcccgcgaggccgagaatgtgaccatgggatgtgccgagggctgcagcttcagcgagaacatcaccgtgcccgacaccaaagtgaacttctacacctggaagagaatggacgtgggccagcaggccgtggaagtgtggcagggactggccctgctgtctgaggccatcctgagaggacaggctctgctggccaacagcagccagcctagcgaaaccctgcagctgcacgtggacaaggccgtgtcctccctgagaagcctgaccagcctgctgagagcactgggagcccagaaagaggccacctctctgcctgaggccacatctgccgcccctctgagaaccttcaccgtggacaccctgtgcaagctgttccggatctacagcaacttcctgcggggcaagctgaccctgtacaccggcgaggcttgtcggagaggcgacagatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - protein, 20 aa, Felis catus
MYRMQLLSCIALSLALVTNS

(19) United States
(12) Patent Application Publication
(10) Pub. No.: US 2023/0355712 A1
(43) Pub. Date: Nov. 9, 2023

(54) COMPOSITION AND USES THEREOF

(71) Applicant: ScoutBio, Inc., Philadelphia, PA (US)

(72) Inventors: Anne TRAAS, Philadelphia, PA (US); Matthew J. WILSON, Philadelphia, PA (US)

(21) Appl. No.: 18/029,010

(22) PCT Filed: Sep. 24, 2021
(86) PCT No.: PCT/US2021/052023

§ 371 Date: Mar. 28, 2023

Related U.S. Application Data
(60) Provisional application No. 63/084,490, filed on Sep. 28, 2020.

(51) Int. Cl. A61K 38/18; A61K 48/00; A61P 7/06; C12N 15/86

(52) U.S. Cl. CPC A61K 38/1816; A61K 48/005; A61P 7/06; C12N 15/86

(57) ABSTRACT

The present disclosure relates generally to compositions and methods for treating feline subjects. An adeno-associated viral vector is provided which includes a nucleic acid molecule comprising a nucleic acid sequence encoding feline erythropoietin (EPO).

### FIELD

This disclosure relates generally to compositions and methods for treating feline subjects, in particular feline subjects who are, or are at risk of becoming, anaemic.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is SCTB-008-01WO_ST25. The text file is 14 KB, created on Sep. 24, 2021, and is being submitted in electronic form herewith.

### BACKGROUND

Erythropoietin (EPO), also known as haematopoietin or haemopoietin, is a hormone made predominantly within the peritubular cells of the kidney, in response to cellular hypoxia. EPO is also produced in the liver, primarily during fetal and perinatal period, but renal synthesis predominates in adulthood. It acts on the bone marrow, stimulating erythropoiesis, to compensate for normal red blood cell turnover (red blood cell homeostasis). Erythropoietin also controls apoptosis (programmed cell death) of mature red blood cells.

Renal disease typically reduces EPO production. In humans, the management of anaemia in chronic kidney disease has been revolutionized by the development of recombinant human EPO (Epoetin). Many of the symptoms that have been ascribed to chronic kidney disease, such as fatigue, lethargy, somnolence and shortness of breath, which all impact unfavorably on quality of life, are resolved or markedly improved when anaemia is corrected. EPO has also been used for the treatment of anaemic conditions resulting from other diseases, such as myelodysplasia, and from chemotherapy. However, there is are risks associated with EPO therapies, including myocardial infarction, stroke and venous thromboembolism. These risks can increase when EPO treatment results in perturbed red blood cell homeostasis or polycythemia; that is, excessive haemoglobin levels (typically greater than 11-12 g/dL in humans).

Anaemia, including anaemia resulting from chronic kidney disease, also affects animals, including feline. There are over 2 million cats that suffer from chronic kidney disease (CKD), noting that companion animals such as cats and dogs with CKD-related renal failure will typically present with similar pathophysiological features as we see in human CKD, including anaemia that is attributed, at least in part, to insufficient levels of EPO. Existing treatments for companion animals with anaemia include the administration of human recombinant EPO. However, as animals will typically develop an immune response to the recombinant EPO, long term treatment is generally ineffective. Therefore, there remains an urgent need for an improved therapeutic regimen for prolonged delivery of EPO in a subject in need thereof, particularly for feline.

### SUMMARY

In an aspect disclosed herein, there is provided a method for promoting red blood cell homeostasis in a feline subject with anaemia, the method comprising administering to a subject in need thereof a composition comprising a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the composition is administered to the subject at a dose effective to promote red blood cell homeostasis in the feline subject, wherein the dose is about 2×10⁹ genome copies per kg body weight (gc/kg) or less. In an embodiment, the dose is about 1×10⁹ genome copies per kg body weight (gc/kg) or less.

The present disclosure also extends to use of a recombinant adeno-associated virus (rAAV) comprising an AAV capsid in the manufacture of a medicament for promoting red blood cell homeostasis in a feline subject with anaemia, wherein the AAV capsid comprises a nucleic acid sequence encoding feline erythropoietin (EPO) and an expression control sequence that directs expression of the EPO in the subject, wherein the rAAV is formulated for administration at a dose effective to promote red blood cell homeostasis in the feline subject, wherein the dose is about 2×10⁹ genome copies per kg body weight (gc/kg) or less. In an embodiment, the dose is about 1×10⁹ genome copies per kg body weight (gc/kg) or less.

In another aspect disclosed herein, there is provided a pharmaceutical composition for use in promoting red blood cell homeostasis in a feline subject with anaemia, wherein the composition comprises a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the rAAV is formulated for administration at a dose effective to promote red blood cell homeostasis in the feline subject, wherein the dose is about 2×10⁹ genome copies per kg body weight (gc/kg) or less. In an embodiment, the dose is about 1×10⁹ genome copies per kg body weight (gc/kg) or less.

The recombinant vectors described herein can be used in a regimen for treating chronic kidney disease and other conditions characterized by an insufficient number of circulating red blood cells (i.e., anaemia).

In another aspect disclosed herein, there is provided a unit dosage form comprising the recombinant adeno-associated virus (rAAV) described herein, wherein the dosage form comprises the rAAV in an amount of from about 1.0×10⁹ to about 5.0×10⁹ genome copies.

The present disclosure also extends to kits comprising the composition or dosage form described herein.

Other aspects and advantages of the invention will be readily apparent from the following detailed description of the invention.

### BRIEF DESCRIPTION OF THE DRAWINGS

FIG. 1 shows the effect of AAV1 expressing feline EPO (AAV1-EPO) on the hematocrit (HCT) in normal laboratory cats. AAV1-EPO was administered at 1.9×10⁸, 6.0×10⁸, 1.9×10⁹ and 6.0×10⁹ gene copy/kg body weight (gc/kg; n=6 per dosage group). Control animals received phosphate buffered saline (PBS) as placebo. Maximum increase in HCT in any individual cat was 15 points (%). In normal healthy cats, doses 1.9×10⁹ and 6.0×10⁹ gc/kg showed a significant and consistent increase in the group mean HCT. 1.9×10⁸ gc/kg and 6.0×10⁸ did not show any significant effect on mean HCT when compared to placebo. Y axis—hematocrit (HCT %); X-axis—days following administration of AAV1-EPO.

FIG. 2 shows the effect of AAV1-EPO at 3.0×10⁹ gc/kg on the hematocrit (HCT) in three cats with CKD-associated anaemia. Y axis—hematocrit (HCT %); X-axis—days following administration of AAV1-EPO at Day 0.

FIG. 3 shows red cell homeostasis in CKD-associated anaemic cats following administration of AAV1-EPO at a dosage range from about 2.0×10⁸ to about 6.0×10⁸ gc/kg. No cats that received treatment with AAV1-EPO required treatment for polycythemia. Y axis—hematocrit or packed cell volume (PCV %); X-axis—days following administration of AAV1-EPO at day 0 (Baseline). * p<0.05 compared to Baseline (paired T-test); error bars indicate standard deviation; n=15, 15, 14, 13, 10, 9 at noted time points, respectively.

FIG. 4 shows an exemplary amino acid sequence of feline EPO (SEQ ID NO:1). The leader sequence is underlined. A codon optimized nucleotide sequence that encodes the feline EPO proprotein of SEQ ID NO:1 is shown in SEQ ID NO:2. An expression cassette for generating a viral vector contains the feline EPO construct sequences flanked by packaging signals of the viral genome and other expression control sequences is shown in SEQ ID NO:3.

FIG. 5 shows the AAV1-feline EPO expression vector encoding feline EPO proprotein (GenBank Accession No. AFN85670.1), the chicken beta-actin promoter and the partial CMV immediate early enhancer. The expression construct is flanked by 5′ and 3′ AAV inverted terminal repeats (ITR). The feline EPO expression construct was then packaged in an AAV serotype 1 (AAV1) capsid by triple transfection and iodixanol gradient purification and titred by Taqman quantitative PCR.

FIG. 6 shows the survival of cats treated with SB-001—from diagnosis of Stage 4 chronic kidney disease to death (n=9). Median survival time was 64 days.

### DETAILED DESCRIPTION

The terminology used herein is for the purpose of describing particular embodiments only and is not intended to be limiting. Unless defined otherwise, all technical and scientific terms used herein have the same meanings as commonly understood by one of ordinary skill in the art to which the present disclosure belongs. Any materials and methods similar or equivalent to those described herein can be used to practice the present invention. Practitioners may refer to Sambrook et al. (1989) Molecular Cloning: A Laboratory Manual, 2nd ed., Cold Spring Harbor Press, Plainsview, N.Y., and Ausubel et al. (1999) Current Protocols in Molecular Biology (Supplement 47), John Wiley & Sons, New York, Murphy et al. (1995) Virus Taxonomy Springer Verlag:79-87, for definitions and terms of the art and other methods known to the person skilled in the art.

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. For example, reference to “a vector” includes a single vector, as well as two or more vectors; reference to “a cell” includes one cell, as well as two or more cells; and so forth.

Nucleotide and amino acid sequences are referred to by sequence identifier numbers (SEQ ID NO:). The SEQ ID NOs correspond numerically to the sequence identifiers <400>1, <400>2, etc. A summary of sequence identifiers is provided herein.

The present invention is predicated, at least in part, on the inventors' surprising finding that a dose of an adenovirus vector that is adapted to drive the expression of recombinant feline EPO and is otherwise sufficient to increase the haematocrit or packed cell volume in a healthy feline subject, is unexpectedly detrimental or fatal to a feline subject with anaemia. The present inventors have unexpectedly found that red blood cell homeostasis can nevertheless be restored in an anaemic feline subject when the feline EPO-expressing adenovirus vector is administered to the anaemic feline subject at a lower dose that would not be expected to increase the haematocrit or packed cell volume in a healthy feline subject. Advantageously, the inventors also found that the low dose treatment regimen described herein minimizes the risk of polycythemia in the anaemic animals.

Accordingly, in an aspect disclosed herein, there is provided a method for promoting red blood cell homeostasis in a feline subject with anaemia, the method comprising administering to a subject in need thereof a composition comprising a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the composition is administered to the subject at a dose effective to promote red blood cell homeostasis in the feline subject, wherein the dose is about 2×10⁹ genome copies per kg body weight (gc/kg) or less.

### Red Blood Cell Homeostasis

The phrase “red blood cell homeostasis”, as used herein, refers to the physiological maintenance of a level of circulating red blood cells in a subject that is understood to be within the acceptable or normal limits for a healthy subject of that species. For example, in feline subjects, a normal level of circulating red blood cells is typically within the range of from about 28.2% to about 50% (haematocrit or packed cell volume).

A hematocrit is calculated as the product of the mean cell volume (MCV) and the red blood cell (RBC) count, both of which can be directly measured by a suitable analyzer, where HCT=(MCV×RBC count)+10. Factors that may falsely increase or decrease the MCV (e.g. storage of RBC may result in RBC swelling with an increased MCV) or RBC count (e.g. hemolysis) can affect the HCT, but not necessarily the PCV. Packed cell volume (PCV) is typically measured by centrifuging a blood sample in a microhematocrit tube in a microhematocrit centrifuge and is taken as the height of the red cell column in a microhematocrit tube after centrifugation as a percentage of the total height of the sample. Unlike HCT, PCV measurements may be affected by plasma trapping and how the red blood cells pack within the column. Both HCT and PCV are expressed as a % of blood (SI units are typically LL); that is, %÷100=L/L. There are varying reports of HCT/PCV values in normal (healthy) feline subjects, although those values typically range from about 28.2% to about 50%. By contrast, anaemic feline subjects will typically have a HCT/PCV of below 28.2%.

Anaemia is typically defined as a deficiency in the number or quality of red blood cells or haemoglobin. Anaemia is caused by a number of different factors, examples of which include blood loss (e.g., trauma and gastrointestinal bleeding), decreased red blood cell production (e.g., iron deficiency, vitamin B12 deficiency, malnutrition, thalassemia, and neoplasms of the bone marrow), and increased red blood cell breakdown (e.g., genetic conditions such as clotting diseases, parasitic infections (e.g., fleas, ticks, hookworms, Mycoplasma haemofelis) and autoimmune diseases). In an embodiment disclosed herein, a feline subject will be characterised as anaemic (i.e., as having anaemia) where the feline subject has a haematocrit or pack cell volume of less than about 29%, preferably less than about 28.2% PCV. In an embodiment, the feline subject with anaemia has a PCV of about 28% or less. In yet another embodiment, the feline subject with anaemia has a PCV in a range of about 10% to about 28%. In an embodiment, the feline subject with anaemia has a PCV in a range of about 22% to about 28%. In an embodiment, the feline subject with anaemia has a PCV of about 22.5%.

In an embodiment, the anaemia is attributed, at least in part, to the use of medications, illustrative examples of which include FIV/feline AIDS treatments and cancer therapeutics, including chemotherapy. In an embodiment, the anaemia is attributed, at least in part, to a medical condition, illustrative examples of which include cancer, FIV/AIDS, rheumatoid arthritis, Crohn's disease and other chronic inflammatory diseases and dysfunctional bone marrow (e.g., aplastic anaemia, leukemia, myelodysplasia or myelofibrosis), multiple myeloma, myeloproliferative disorders and lymphoma, hemolytic anaemia, sickle cell anaemia and thalassemia. In an embodiment, the anaemia is attributed to chronic kidney disease. In an embodiment, the anaemia is associated with chronic kidney disease.

In some embodiments, it may be desirable to treat a feline subject that is at risk of developing anaemia, for example, where a feline subject has been diagnosed with a disease or conditions that is likely to promote an anaemic state but otherwise has a HCT/PCV within the normal range. In those circumstances, the methods described herein may be used to prevent anaemia or restore red blood cell homeostasis once the feline subject becomes anaemic. Thus, also contemplated herein is a method for promoting red blood cell homeostasis in a feline subject that is at risk of developing anaemia, the method comprising administering to a subject in need thereof a composition comprising a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the composition is administered to the subject at a dose effective to promote red blood cell homeostasis in the feline subject, wherein the dose is about 1×10⁹ genome copies per kg body weight (gc/kg) or less.

It will be understood by persons skilled in the art that the methods described herein will have promoted (i.e., restored) red blood cell homeostasis in the feline subject where, following administration of the AAV vector, there is an increase in the HCT/PVC in the subject to a value within the acceptable or normal range for a healthy subject of that species.

In an embodiment, the dose is effective to increase the PCV in the feline subject by about 5 to about 30 PCV points by day 70 following administration of the composition. In another embodiment, the dose is effective to increase the PCV in the feline subject by about 10 to about 20 PCV points by day 70 following administration of the composition. In yet another embodiment, the dose is effective to increase the PCV in the feline subject by about 15 PCV points by day 70 following administration of the composition.

In an embodiment, the dose is effective to increase the PCV in the feline subject to a value from about 30% to about 55% by day 70 following administration of the composition. In another embodiment, the dose is effective to increase the PCV in the feline subject to a value from about 30% to about 35% by day 70 following administration of the composition. In yet another embodiment, the dose is effective to increase the PCV in the feline subject to a value from about 33% to about 35% by day 70 following administration of the composition.

As noted elsewhere herein, the present inventors have surprisingly found that the dosage regimen described herein promotes or otherwise restores red blood cell homeostasis whilst avoiding polycythemia (excessive or abnormally high red blood cell levels). Thus, in an embodiment, the feline subject does not develop polycythemia by day 70 following administration of the composition. In another embodiment, the feline subject does not require treatment for polycythemia, preferably does not require treatment for polycythemia by day 70 following administration of the composition.

In an embodiment, the composition is administered intramuscularly to the feline subject as a unit dose of about 2×10⁹ genome copies per kg body weight (gc/kg) or less. In an embodiment, the composition is administered intramuscularly to the feline subject as a unit dose of about 1×10⁹ genome copies per kg body weight (gc/kg) or less. In an embodiment, the composition is administered intramuscularly to the feline subject as a unit dose of from about 1.0×10⁹ to about 5.0×10⁹ genome copies. In an embodiment, the composition is administered intramuscularly to the feline subject as a unit dose of from about 1.2×10⁹ to about 5.0×10⁹ genome copies.

The present disclosure also extends to use of a recombinant adeno-associated virus (rAAV) comprising an AAV capsid in the manufacture of a medicament for promoting red blood cell homeostasis in a feline subject with anaemia, wherein the AAV capsid comprises a nucleic acid sequence encoding feline erythropoietin (EPO) and an expression control sequence that directs expression of the EPO in the subject, wherein the rAAV is formulated for administration at a dose effective to promote red blood cell homeostasis in the feline subject, wherein the dose is about 2×10⁹ genome copies per kg body weight (gc/kg) or less. In an embodiment, the dose is about 1×10⁹ genome copies per kg body weight (gc/kg) or less. In an embodiment, the composition is formulated for intramuscular administration to the feline subject as a unit dose of about 2×10⁹ genome copies per kg body weight (gc/kg) or less. In an embodiment, the composition is formulated for intramuscular administration to the feline subject as a unit dose of about 1×10⁹ genome copies per kg body weight (gc/kg) or less. In an embodiment, the composition is formulated for intramuscular administration to the feline subject as a unit dose of from about 1.0×10⁹ to about 5.0×10⁹ genome copies. In an embodiment, the composition is formulated for intramuscular administration to the feline subject as a unit dose of from about 1.2×10⁹ to about 5.0×10 genome copies.

In another aspect disclosed herein, there is provided a pharmaceutical composition for use in promoting red blood cell homeostasis in a feline subject with anaemia, wherein the composition comprises a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the rAAV is formulated for administration at a dose effective to promote red blood cell homeostasis in the feline subject, wherein the dose is about 2×10⁹ genome copies per kg body weight (gc/kg) or less. In an embodiment, the dose is about 1×10⁹ genome copies per kg body weight (gc/kg) or less. In an embodiment, the composition is formulated for intramuscular administration to the feline subject as a unit dose of about 2×10⁹ genome copies per kg body weight (gc/kg) or less. In an embodiment, the composition is formulated for intramuscular administration to the feline subject as a unit dose of about 1×10 genome copies per kg body weight (gc/kg) or less. In an embodiment, the composition is formulated for intramuscular administration to the feline subject as a unit dose of from about 1.0×10⁹ to about 5.0×10⁹ genome copies. In an embodiment, the composition is formulated for intramuscular administration to the feline subject as a unit dose of from about 1.2×10⁹ to about 5.0×10⁹ genome copies.

In another aspect disclosed herein, there is provided a method of treating an anaemic feline subject with chronic kidney disease, the method comprising administering to the subject a composition comprising a recombinant adeno-associated virus (rAAV) comprising an AAV1 capsid, wherein the AAV1 capsid comprises a nucleic acid sequence encoding feline erythropoietin (EPO) having an amino acid sequence of SEQ ID NO:1, and an expression control sequence that directs expression of the EPO in the subject, wherein the composition is administered intramuscularly at a dose of (i) about 3.65×10⁹ genome copies to a feline subject weighing from 2 to 6 kg or (ii) about 7.30×10⁹ genome copies to a feline subject weighing more than 6 kg.

In another aspect disclosed herein, there is provided use of a recombinant adeno-associated virus (rAAV) comprising an AAV1 capsid in the manufacture of a medicament for treating an anaemic feline subject with chronic kidney disease, wherein the AAV1 capsid comprises a nucleic acid sequence encoding a feline erythropoietin (EPO) having an amino acid sequence of SEQ ID NO:1, and an expression control sequence that directs expression of the EPO in the subject, wherein the rAAV is formulated for intramuscular administration at a dose of (i) about 3.65×10⁹ genome copies to a feline subject weighing from 2 to 6 kg or (ii) about 7.30×10⁹ genome copies to a feline subject weighing more than 6 kg.

In another aspect disclosed herein, there is provided a pharmaceutical composition for use in treating an anaemic feline subject with chronic kidney disease, wherein the composition comprises a recombinant adeno-associated virus (rAAV) comprising an AAV1 capsid, wherein the AAV1 capsid comprises a nucleic acid sequence encoding a feline erythropoietin (EPO) having an amino acid sequence of SEQ ID NO:1, and an expression control sequence that directs expression of the EPO in the subject, wherein the rAAV is formulated for intramuscular administration at a dose of (i) about 3.65×10⁹ genome copies to a feline subject weighing from 2 to 6 kg or (ii) about 7.30×10⁹ genome copies to a feline subject weighing more than 6 kg.

In another aspect disclosed herein, there is provided a method of treating an anaemic feline subject with chronic kidney disease, the method comprising administering to the subject a composition comprising a recombinant adeno-associated virus (rAAV) comprising an AAV1 capsid, wherein the AAV1 capsid comprises a nucleic acid sequence encoding feline erythropoietin (EPO) having an amino acid sequence of SEQ ID NO:1, and an expression control sequence that directs expression of the EPO in the subject, wherein the composition is administered intramuscularly at a dose of (i) about 3.65×10⁹ genome copies to a feline subject weighing from 2 to 6 kg or (ii) about 7.30×10⁹ genome copies, administered in the form of two doses each comprising about 3.65×10⁹ genome copies, to a feline subject weighing more than 6 kg.

In another aspect disclosed herein, there is provided use of a recombinant adeno-associated virus (rAAV) comprising an AAV1 capsid in the manufacture of a medicament for treating an anaemic feline subject with chronic kidney disease, wherein the AAV1 capsid comprises a nucleic acid sequence encoding a feline erythropoietin (EPO) having an amino acid sequence of SEQ ID NO:1, and an expression control sequence that directs expression of the EPO in the subject, wherein the rAAV is formulated for intramuscular administration at a dose of (i) about 3.65×10⁹ genome copies to a feline subject weighing from 2 to 6 kg or (ii) about 7.30×10⁹ genome copies, administered in the form of two doses each comprising about 3.65×10⁹ genome copies, to a feline subject weighing more than 6 kg.

In another aspect disclosed herein, there is provided a pharmaceutical composition for use in treating an anaemic feline subject with chronic kidney disease, wherein the composition comprises a recombinant adeno-associated virus (rAAV) comprising an AAV1 capsid, wherein the AAV1 capsid comprises a nucleic acid sequence encoding a feline erythropoietin (EPO) having an amino acid sequence of SEQ ID NO:1, and an expression control sequence that directs expression of the EPO in the subject, wherein the rAAV is formulated for intramuscular administration at a dose of (i) about 3.65×10⁹ genome copies to a feline subject weighing from 2 to 6 kg or (ii) about 7.30×10⁹ genome copies, administered in the form of two doses each comprising about 3.65×10⁹ genome copies, to a feline subject weighing more than 6 kg.

### Erythropoietin (EPO)

EPO is a glycoprotein hormone produced predominantly in the kidney. Human EPO is heavily glycosylated and has a molecular mass of about 30 kDa, 40% of which is derived from its carbohydrate component. EPO promotes survival of EPO-dependent colony-forming unit-erythroid (CFU-E) cells and erythroblasts that have not yet begun to synthesize hemoglobin. Upon ligand binding, the EPO receptor (EpoR), which lacks intrinsic catalytic function and is hypoxia-inducible, associates with tyrosine kinase Janus kinase 2 (JAK2), which in turn phosphorylates EpoR and provides multiple docking sites for signal-transducing proteins that contain src homology 2 (SH2) domains. Signaling at the EpoR occurs through multiple pathways that include the signal transduction and activator of transcription (STAT) 5 pathway, the phosphatidylinositol-3-kinase/protein kinase B (PI-3K/AKT) and mitogen-associated protein kinase/extracellular signal-related kinase (MAPK/ERK) pathways, as well as protein kinase C. A negative feedback system, in which tissue oxygenation controls EPO production and EPO controls red blood cell (RBC) production, provides homeostasis in oxygen delivery to body tissues (Koury and Bondurant, 1991; Am J Kidney Dis.; 18(4 Suppl 1):20-3). Although EPO may have some effect on mitosis in early erythroid progenitor cells, its control of RBC production appears to occur in later stages of erythroid cell development, where it prevents programmed cell death.

As noted by H. Franklin Bunn in the 2013 paper, “Erythropoietin” (ColdSpring Harb Perspect Med.; 3(3):a011619), erythropoiesis normally proceeds in humans and other mammals at a low basal rate, replacing senescent red blood cells with young reticulocytes. In humans, red cell production can be enhanced as much as eightfold above the baseline rate in a variety of clinical settings including hemorrhage, hemolysis, and other types of stress that impair the oxygenation of arterial blood or the delivery of oxygen to the tissues. During fetal development, EPO is produced mainly in the liver, although following birth, the kidney accounts for ˜80% of EPO production.

Human Epo messenger RNA (mRNA) encodes a 193-residue polypeptide. Following cleavage of a canonical leader sequence in the endoplasmic reticulum and post-translational glycosylation, a 166-residue polypeptide is released. The primary structure of rhEpo was shown to be identical to that of the endogenous hormone, except for the in vivo post-translational cleavage of an arginine residue at the C-terminus. EPO circulates in plasma with a plasma half-life of about 7-8 hours and binds to high-affinity (˜100 pM) receptors present in relatively small numbers (˜1000/cell) on the surface of erythroid progenitor cells (CFUe) in the bone marrow.

EPO is expressed in vivo as a propeptide or precursor protein, with the leader sequences that shares homology across different species (see, e.g., Wen et al. (1993), Blood, 82(5):1507-1516). The amino acid and nucleotide sequences of feline EPO will be familiar to persons skilled in the art, illustrative examples of which are described in GenBank Accession Nos. NM_001009269, U00685, AFN85670, AAA18282, Wen et al. (1993, Blood, 82(5):1507-1516), Walker et al. (2005, AJVR, 66(3):450-456), Beall et al. (2000, Gene Therapy, 7:534-539) and WO 2017/040524, the contents of which are incorporated herein by reference in their entirety.

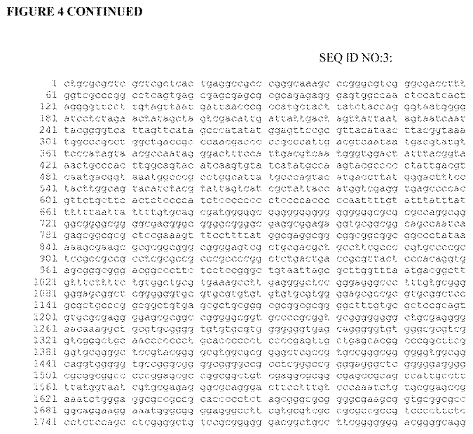

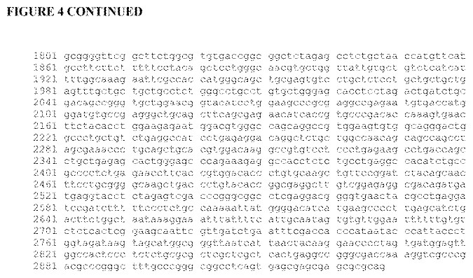

In an embodiment, the nucleic acid sequence encoding the feline EPO encodes a feline EPO proprotein that comprises, consists or consists essentially of the amino acid sequence of SEQ ID NO:1, or an amino acid sequence that has at least 70% sequence identity thereto. The feline EPO proprotein of SEQ ID NO:1 sequence is shown in FIG. 4, noting that the mature feline EPO protein begins at amino acid position 27 of SEQ ID NO:1.

In an embodiment, the feline EPO is encoded by a nucleic acid sequence comprising, consisting or consisting essentially of the nucleic acid sequence of SEQ ID NO: 2, or a nucleotide acid sequence that has at least 70% sequence identity thereto.

In an embodiment, an expression cassette for generating a viral vector contains the feline EPO construct sequences flanked by packaging signals of the viral genome and other expression control sequences that comprise, consist or consist essentially of the nucleic acid sequence of SEQ ID NO:3.

In an embodiment, an expression cassette encoded by a nucleic acid sequence comprising, consisting or consisting essentially of the nucleic acid sequence of SEQ ID NO:3, or a nucleotide acid sequence that has at least 70% sequence identity thereto.

In an embodiment, the feline EPO is a full length feline EPO protein. By “full length” is meant that the feline EPO encoded by the nucleic acid sequence described herein comprises an amino acid sequence that shares 100% sequence identity to a native feline EPO protein; that is, to a naturally-occurring feline EPO protein.

Functional variants of feline EPO are also contemplated herein. As used herein, the term “functional variant” refers to an amino acid sequence that shares at least some sequence identity to native feline EPO whilst also retaining at least some of the biological activity that is typically ascribed to the native feline EPO protein, such as the ability to increase red blood cell production in vivo. Functional variants may therefore suitably extend to functional fragments of a native feline EPO protein. Methods of identifying functional variants of feline EPO, as described herein, will be familiar to persons skilled in the art, illustrative examples of which are described elsewhere herein.

In an embodiment, functional variants of EPO include variants which may include up to about 10% variation from an EPO nucleic acid or amino acid sequence described herein or known in the art, which retain the function of the wild type sequence. As used herein, by “retain function” it is meant that the nucleic acid or amino acid functions in the same way as the wild type sequence, although not necessarily at the same level of expression or activity. For example, in one embodiment, a functional variant has increased expression or activity as compared to the wild type sequence. In another embodiment, the functional variant has decreased expression or activity as compared to the wild type sequence. In one embodiment, the functional variant has 10%, 20%, 30%, 40%, 50%, 60%, 70%, 80%, 90% or greater increase or decrease in expression or activity as compared to the wild type sequence.

In another embodiment, functional variants of EPO include variants which may include up to about 20% variation from an EPO nucleic acid or amino acid sequence described herein or known in the art, which retain the function of the wild type sequence. In one embodiment, functional variants of EPO include variants which may include up to about 30% variation from an EPO nucleic acid or amino acid sequence described herein or known in the art, which retain the function of the wild type sequence.

In an embodiment, the feline EPO comprises, consists, or consists essentially of the amino acid sequence of SEQ ID NO:1, or an amino acid sequence having at least 70%, preferably at least 75%, preferably at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity thereto.

In an embodiment, the feline EPO sequence is encoded by the nucleic acid sequence of SEQ ID NO:2 or a nucleic acid sequence having at least 70%, preferably at least 75%, preferably at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity thereto.

In an embodiment, the expression cassette for generating a viral vector containing the feline EPO construct sequences flanked by packaging signals of the viral genome and other expression control sequences is encoded by the nucleic acid sequence of SEQ ID NO:3 or a nucleic acid sequence having at least 70%, preferably at least 75%, preferably at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity thereto.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated, typically in a host cell, to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

Reference to “at least 70%/” includes 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of the recited sequences, including SEQ ID NOs:1, 2, and 3 after optimal alignment or best fit analysis.

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any suitable method known to persons skilled in the art. Reference also may be made to the BLAST family of programs as, for example, disclosed by Altschul et al. (1997) Nucl. Acids. Res. 25:3389. A detailed discussion of sequence analysis can also be found in Unit 19.3 of Ausubel et al. (1994-1998) In: Current Protocols in Molecular Biology, John Wiley & Sons Inc.

The term “sequence identity”, as used herein, refers to the extent that sequences are identical or structurally similar on a nucleotide-by-nucleotide basis or an amino acid-by-amino acid basis over a window of comparison. For example, two or more peptide sequences may be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). Suitable methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm. A “percentage of sequence identity” may therefore be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. For example, “sequence identity” is the “match percentage” calculated by the DNASIS computer program (Version 2.5 for windows; available from Hitachi Software engineering Co., Ltd., South San Francisco, California, USA) using standard defaults as used in the reference manual accompanying the software.

The term “sequence identity”, as used herein, includes exact identity between compared sequences at the nucleotide or amino acid level. Sequence identity, as herein described, typically relates to the percentage of amino acid residues in the candidate sequence that are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology.

The present disclosure also extends to non-exact identity (i.e., similarity) of sequences at the nucleotide or amino acid level where any difference(s) between sequences are in relation to amino acids (or in the context of nucleotides, amino acids encoded by said nucleotides) that are nevertheless related to each other at the structural, functional, biochemical and/or conformational levels. For example, where there is non-identity (similarity) at the amino acid level, “similarity” includes amino acids that are nevertheless related to each other at the structural, functional, biochemical and/or conformational levels. In an embodiment, nucleotide and sequence comparisons are made at the level of identity rather than similarity. For example, leucine may be substituted for an isoleucine or valine residue. This may be referred to as a conservative substitution. In an embodiment, the amino acid sequences may be modified by way of conservative substitution of any of the amino acid residues contained therein, such that the modification has no or negligible effect on the functional activity of the modified polypeptide when compared to the unmodified polypeptide.

In an embodiment disclosed herein, the nucleic acid sequence is codon optimized.

In an embodiment, the feline EPO comprises, consists or consists essentially of a functional variant having at least 70% sequence identity to a feline EPO protein yet retains the same, or substantially the same, function of the wild type feline EPO protein. The sequence on which the feline EPO variant is based may, in some embodiments, include the propeptide leader sequence (e.g., as shown in SEQ ID NO:1; FIG. 4). In another embodiment, the feline EPO variant described herein comprises, consists or consists essentially of only the mature peptide (e.g., amino acid residues 27-192 of SEQ ID NO:1). As used herein, the term “retain function” means the functional variant functions in the same way, or substantially the same way, as a wild type feline EPO protein, although not necessarily at the same level of expression or activity. For example, a functional variant may have increased expression or activity as compared to a wild type feline EPO protein. Alternatively, the functional variant may have decreased expression or activity as compared to a wild type feline EPO sequence. In an embodiment, the functional variant has at least 10%, preferably at least 20%, preferably at least 30%, preferably at least 40%, preferably at least 50%, preferably at least 60%, preferably at least 70%, preferably at least 80%, preferably at least 90% or more preferably at least 95% of the expression or activity of the wild type feline EPO protein.

In an embodiment disclosed herein, the nucleic acid sequence encoding the feline EPO comprises a heterologous leader sequence. The term “heterologous”, when used with reference to an amino acid or nucleic acid sequence, typically indicates that the amino acid or nucleic acid sequence comprises two or more sequences or subsequences which are not found in the same relationship to each other in nature. For example, an expression cassette is typically recombinantly produced, having two or more sequences from unrelated genes arranged to make a new functional nucleic acid. In one embodiment, the leader sequence may be from a different gene: that is, other than a gene encoding EPO. In another embodiment, the leader sequence is derived from a species other than feline. Suitable leader sequences will be familiar to persons skilled in the art, illustrative examples of which include a signal sequence of a cytokine (e.g., IL-2, IL12, IL18), an immunoglobulin, insulin, albumin, β-glucuronidase, an alkaline protease, fibronectin secretory signal peptides, or sequences from tissue specific secreted proteins. In an embodiment, the leader sequence encodes an IL-2 signal peptide (e.g., SEQ ID NO:4: M Y R M Q L L S C I A L S L A L V T N S). In an embodiment, the leader sequence is the endogenous leader sequence from the EPO propeptide (e.g., amino acid residues 1-26 of SEQ ID NO:1).

The terms “derived” or “derived from” are used interchangeably herein to mean that the sequence (amino acid or nucleotide sequence) is sourced from a feline subject or shares the same sequence as the protein or nucleotide sequence sourced from a feline subject. For example, a propeptide sequence that is “derived from” a feline may share the same sequence (or a functional variant thereof, as defined herein) as the same propeptide sequence as expressed in a feline. However, the specified nucleic acid or amino acid need not actually be sourced from a feline. Various techniques will be known to persons skilled in the art that can be used to produce a desired sequence, including mutagenesis of a similar protein (e.g., a homolog) or artificial (recombinant) production of a nucleic acid or amino acid sequence. The derived nucleic acid or amino acid will typically retains the same, or substantially the same, function of the same nucleic acid or amino acid in the species from which it is derived, regardless of actual source of the derived sequence.

The present disclosure also extends to functional variants of feline EPO in which one or more amino acid substitutions have been made to the wild type feline EPO sequence (e.g., SEQ ID NO:1). In an embodiment, the one or more amino acid substitutions comprise one or more conservative amino acid substitutions, illustrative examples of which are described elsewhere herein.

The term “amino acid substitution” and the like are intended to encompass modification of an amino acid sequence by replacement of an amino acid with another, substituting, amino acid residue. The substitution may be a conservative amino acid substitution, as described elsewhere herein. In some embodiments, the substitution may be a non-conservative substitution. The term “conservative”, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence.

Reference to “one or more” herein is intended to encompass the individual embodiments of, for example, 1, 2, 3, 4, 5, 6, or more.

As noted elsewhere herein, the nucleic acid sequence encoding the feline EPO may be codon optimized. For instance, where a functional variant of the feline EPO peptide is desired, the coding sequences for these functional variants may be generated using site-directed mutagenesis of the wild-type nucleic acid sequence. Web-based or commercially available computer programs, as well as service based companies may be used to back translate the amino acids sequences to nucleic acid coding sequences, including both RNA and/or cDNA. See, e.g., backtranseq by EMBOSS, http://www.ebi.ac.uk/Tools/st/; Gene Infinity (http://www.geneinfinity.org/sms-/sms_backtranslation.html); ExPasy (http://www.expasy.org/tools/). In one embodiment, the RNA and/or cDNA coding sequences are designed for optimal expression in the subject species for which administration is ultimately intended, as discussed herein. Thus, in one embodiment, the coding sequences are designed for optimal expression in a feline.

The coding sequences may be designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Publication No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In one embodiment, the nucleic acid sequence encoding EPO is a codon optimized sequence encoding any of the EPO peptides described herein, including sequences sharing at least 90% identity with the described sequence. In one embodiment, the nucleic acid sequence is codon optimized for expression in the subject for which administration is desired.

### Adeno-Associated Viral Vectors

Adeno-associated viral vectors carrying the feline EPO expression constructs have been developed for use in feline subjects. The development and manufacture of a recombinant feline EPO protein as a therapeutic agent would be cost prohibitive when compared to a viral vector mediated system for expression of recombinant EPO in an affected animal. Viral vector therapeutics also have the advantage of convenience, insofar as only a single administration or a lower number of doses may be required to treat the subject, as opposed to requiring recurrent administration of recombinant EPO protein, thereby further improving quality of life. The EPO constructs described herein suitably provide an EPO sequence that is native to the feline subject to be treated, as this will typically reduce or otherwise avoid the risk of the subject developing an immune response to a non-native EPO protein.

By “vector” and “construct”, which is used interchangeably, is meant a nucleic acid molecule, preferably a DNA molecule derived, for example, from a plasmid, bacteriophage, or virus, into which a nucleic acid sequence encoding the feline EPO may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector or construct may be an autonomously replicating vector (i.e., a vector that exists as an extrachromosomal entity), the replication of which is independent of chromosomal replication (e.g., a linear or closed circular plasmid, an extrachromosomal element, a minichromosome, or an artificial chromosome). The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes will be familiar to persons skilled in the art.

In an embodiment, the vector is a recombinant adeno-associated virus (AAV) vector that enables persistent expression of recombinant feline EPO in the subject.

Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61: 447-57 (1990)). These proteins also regulate transcription from endogenous AAV promoters and promoters within helper viruses (see, e.g., Pereira et al., J. Virol., 71: 1079-1088 (1997)). The other Rep proteins modify the function of Rep78 and Rep68. The cap genes encode the capsid proteins VP1, VP2, and VP3. The cap genes are transcribed from the p40 promoter.

Also disclosed herein is an expression construct comprising a nucleic acid sequence encoding the EPO molecule, as described herein, operably linked to one or more regulatory sequences.

As used herein the terms “EPO construct”. “EPO expression construct” and synonyms include the EPO sequence as described herein. The terms “EPO construct”, “EPO expression construct” and synonyms thereof can be used to refer to the nucleic acid sequences encoding the EPO (including the EPO mature protein or propeptide with endogenous or heterologous leader) or the expression products thereof.

The term “construct” is also taken to mean a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Suitable constructs will generally include the necessary elements to direct expression of a nucleic acid sequence of interest that is also contained in the construct, such as, for example, a target nucleic acid sequence or a modulator nucleic acid sequence. Such elements may include control elements or regulatory sequences such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells will be well known to persons skilled in the art. See, for example, Molecular Cloning: A Laboratory Manual, 3^{rd} edition Volumes 1, 2, and 3. J. F. Sambrook, D. W. Russell, and N. Irwin, Cold Spring Harbor Laboratory Press, 2000.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokarvotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

In an embodiment, the nucleic acid sequences encoding the feline EPO constructs described herein are engineered into any suitable genetic element, illustrative examples of which include naked DNA, phage, transposon, cosmid, RNA molecule (e.g., mRNA), episome, etc., which transfers the EPO sequences carried thereon to a host cell, e.g., for generating nanoparticles carrying DNA or RNA, viral vectors in a packaging host cell and/or for delivery to a host cells in subject. In an embodiment, the genetic element is a plasmid. The selected genetic element may be delivered by any suitable method known to persons skilled in the art, illustrative examples of which include by transfection, electroporation, liposome delivery, membrane fusion techniques, high velocity DNA-coated pellets, viral infection and protoplast fusion. The methods used to make such constructs will also be familiar to persons skilled in the art of nucleic acid manipulation, illustrative examples of which include genetic engineering, recombinant engineering, and synthetic techniques. See, e.g., Green and Sambrook, Molecular Cloning: A Laboratory Manual, Cold Spring Harbor Press, Cold Spring Harbor, NY (2012), the entire contents of which is incorporated herein by reference.

As used herein, an “expression construct” refers to a nucleic acid molecule which comprises coding sequences for feline EPO, promoter, and may include other regulatory sequences therefor, which cassette may be engineered into a genetic element and/or packaged into the capsid of a viral vector (e.g., a viral particle). Typically, such an expression cassette for generating a viral vector contains the feline EPO construct sequences described herein flanked by packaging signals of the viral genome and other expression control sequences such as those described herein. Any of the expression control sequences can be optimized for a specific species using techniques known in the art including, e.g., codon optimization, as described elsewhere herein.

The expression cassette typically contains a promoter sequence as part of the expression control sequences. A promoter is a region of nucleic acid that initiates transcription of a particular gene or downstream nucleic acid. Promoters may be located near the transcription start site of genes. Promoters are critical elements of expression cassettes and expression vectors, and may work in conjunction with other regulatory elements, such as enhancers, silencers and insulators to direct the level of transcription of a given gene. As such, different promoters may direct different levels of transcription.

As noted elsewhere herein, the present disclosure relates to the use of an expression construct for the controlled expression of recombinant feline EPO for promoting (or restoring) red blood homeostasis in a feline subject in need thereof. It will be understood that the level of expression of the recombinant feline EPO may be suitably controlled by the type of promoter that is used to drive the expression of the recombinant feline EPO in the feline subject.

It will be understood that, in the context of promoters, the terms “strong” and “weak” are typically used to describe promoters according to their effects on transcription rates and subsequent gene expression, wherein a “strong” promoter typically provides a faster rate of transcription as compared to a “weak” promoter. A promoter may be classified as strong or weak according to its affinity for RNA polymerase (and/or sigma factor); this is typically related to how closely the promoter sequence resembles the ideal consensus sequence for the polymerase. The strength of a promoter may also depend on whether initiation of transcription occurs at that promoter with high or low frequency. Different promoters with different strengths may be used to construct genetic circuits with different levels of gene/protein expression (e.g., the level of expression initiated from a weak promoter is lower than the level of expression initiated from a strong promoter).

As used herein, the term “a strong promoter” refers to a promoter that allows the gene under its control to be expressed at a higher level when compared to a weak promoter. A strong promoter may be naturally occurring, or it may be a modified or synthetic promoter, e.g. a derivative of a naturally occurring promoter. It may thus be native or non-native. The term “strong promoter” will be familiar to persons skilled in the art, illustrative examples of which are widely described in the literature (see, e.g., Schlabeach et al., 2010, PNAS, 107(6):2538-2543 and Bienick et al., 2014, PLoS online, 9(10):e109105, the contents of which are incorporated herein by reference in their entirety). Strong promoters can produce large amounts of transcript and final protein product from the gene of interest. For example, strong promoters can express proteins at a level of at least 1% of the total cellular protein. Preferably, a strong promoter can express proteins at a level of 2, 5, 10, 15, 20, 25, 30, 35, 40, 45 or 50% of the total cellular protein. Accordingly, a promoter may be a strong promoter if it achieves the above expression levels at the selected conditions in the context of a particular host cell and expression system, i.e., it may be a strong promoter for the particular method and reagents used. Preferably, the strong promoter allows expression of the gene of interest such that the protein encoded by the gene of interest can be purified from the cells at quantities of at least 1 milligram per litre. More preferably, at 5 milligrams per litre. Even more preferably, at 10 milligrams per litre. A strong promoter typically drives expression of a coding sequence at a high level, or at about 1/10 transcripts to about 1/100 transcripts to about 1/1,000 transcripts.

Generally, by “weak promoter” is a promoter that drives expression of a coding sequence at a low level as compared to a strong promoter. By “low level” is intended at levels of about 1/10,000 transcripts to about 1/100,000 transcripts. In certain aspects of the present disclosure, the promoter that drives expression of the feline EPO is a weak promoter. That is, it directs lower levels of transcription of the feline EPO than strong promoters. The relative weakness of the promoter may be determined by methods known in the art, such as by methods disclosed in Qin et al. (2010, PloS One, 5(5): e10611; 1-4), the entire contents of which are incorporated herein by reference. Suitable weak promoters will be familiar to persons skilled in the art, illustrative examples of which are widely described in Schlabeach et al. (2010, PNAS, 107(6):2538-2543) and Bienick et al. (2014, PLoS online, 9(10):e109105), the contents of which are incorporated herein by reference in their entirety).

The strength of a promoter, whether “strong” or “weak”, can be assessed when used in an identical vector context (i.e., all other elements of the vector are identical), and when expressed in the same cell type and under the same conditions. In this context, any suitable measure for promoter strength may be used, for example transcript number, or amount of protein produced.

Illustrative examples of suitable strong promoters include Pm promoter, Ptac, Ptrc\ddot{ι}l RNA polymerase promoter (P₇φ1 0), XPL and PBAD and a derivatives of any of ther foregoing. Preferably, the strong promoter allows expression of the recombinant feline EPO in a host cell such that the recombinant feline EPO can be purified from the cells at quantities of at least 1 milligram per litre, more preferably at 5 milligrams per litre or even more preferably at 10 milligrams per litre. A strong promoter will typically drive expression of a coding sequence at a high level, or at about 1/10 transcripts to about 1/100 transcripts to about 1/1,000 transcripts.

Other illustrative examples of strong promoters include the human cytomegalovirus (CMV) promoter, SV40 promoter, EF1a promoter, human β actin promoter, chicken β actin promoter, lac promoter, trp promoter, trc promoter, tac promoter, MPSV, the PR promoter and the PL promoter of lambda phage, viral LTRs including RSV LTR, HIV-1 LTR, HTLV-1 LTR, and the like. Other elements may also be used to enhance expression, such as the HIV tat and TAR system. Other strong promoters include inducible promoters.

Illustrative examples of weak promoters include PCₒN (see, e.g., Dobrynin et al., Nucleic acid Res. Symp. Ser., 7, 365-376, 1980), UBC promoter, and PGK promoter.

In another embodiment, the promoter is a TBG promoter.

In an embodiment, the promoter is a CB7 promoter. CB7 is a chicken β-actin promoter with cytomegalovirus enhancer elements. Alternatively, other liver-specific promoters may be used (see, e.g., The Liver Specific Gene Promoter Database, Cold Spring Harbor, http://rulai.schl.edu/LSPD, alpha 1 anti-trypsin (A1AT); human albumin Miyatake et al., J. Virol., 71:512432 (1997), humAlb; and hepatitis B virus core promoter, Sandig et al., Gene Ther., 3:10029 (1996)). TTR minimal enhancer/promoter, alpha-antitrypsin promoter, LSP (845 nt)25 (requires intron-less scAAV). In one embodiment, the liver-specific promoter thyroxin binding globulin (TBG) is used. Other promoters, such as viral promoters, constitutive promoters, regulatable promoters (see, e.g., WO 2011/126808 and WO 2013/04943), or a promoter responsive to physiologic cues may be used may be utilized in the vectors described herein.

In addition to a promoter, an expression cassette and/or a vector may contain other appropriate transcription initiation, termination, enhancer sequences, efficient RNA processing signals such as splicing and polyadenylation (polyA) signals; TATA sequences; sequences that stabilize cytoplasmic mRNA; sequences that enhance translation efficiency (i.e., Kozak consensus sequence); introns; sequences that enhance protein stability; and when desired, sequences that enhance secretion of the encoded product. The expression cassette or vector may contain none, one or more of any of the elements described herein. Examples of suitable polyA sequences include, e.g., SV40, bovine growth hormone (bGH), and TK polyA. Examples of suitable enhancers include, e.g., the CMV enhancer, the RSV enhancer, the alpha fetoprotein enhancer, the TTR minimal promoter/enhancer, LSP (TH-binding globulin promoter/alpha1-microglobulin/bikunin enhancer), amongst others.

Thus, the level of expression of the feline EPO, and the dose effective to promote red blood cell homeostasis in a feline subject in need thereof, may depend on the type of promoter that is employed, which may determine the dose that is effective to promote or restore red blood cell homeostasis in the feline subject. Thus, in another aspect disclosed herein, there is provided a method for promoting red blood cell homeostasis in a feline subject with anaemia, the method comprising administering to a subject in need thereof a composition comprising a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the composition is administered to the subject at a dose effective to promote red blood cell homeostasis in the feline subject, wherein the dose is effective to increase the PCV of the feline subject by a value from about 5 to about 30 PCV % points, preferably by at least about 15 PCV % points.

In another aspect disclosed herein, there is provided a method for promoting red blood cell homeostasis in a feline subject with anaemia, the method comprising administering to a subject in need thereof a composition comprising a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the composition is administered to the subject at a dose effective to promote red blood cell homeostasis in the feline subject, wherein the dose is effective to provide an increase in PCV in the feline subject that is equivalent to an increase in PCV that would be achieved by a dose of about 2×10⁹ genome copies per kg body weight (gc/kg) or less of an AAV capsid in which expression of the feline EPO is driven by a strong promoter. In an embodiment, the dose is effective to provide an increase in PCV in the feline subject that is equivalent to an increase in PCV that would be achieved by a dose of about 1×10⁹ genome copies per kg body weight (gc/kg) or less of an AAV capsid in which expression of the feline EPO is driven by a strong promoter.

In an embodiment, the expression control sequence comprise a tissue-specific promoter. Suitable tissue-specific promoters will be familiar to persons skilled in the art. In an embodiment, the tissue-specific promoter is a kidney-specific promoter or a muscle-specific promoter. In an embodiment, the tissue-specific promoter is selected from a Nkcc2 promoter, uromodulin promoter, Ksp-cadherin promoter and THP gene promoter.

In an embodiment, the AAV capsid further comprises one or more of an intron, a Kozak sequence, a polyA, and post-transcriptional regulatory elements.

Suitable AAV capsids will be familiar to persons skilled in the art, illustrative examples of which include AAV1, AAV5, AAV6, AAV8, AAVrh64R1, AAV9, AAVrh91, AAVhu.37, AAV3b, AAV3b.AR2.12 and AAVrh10. In an embodiment, the AAV capsid is selected from the group consisting of AAV1, AAV5, AAV6, AAV8, AAVrh64R1, AAV9, AAVrh91, AAVhu.37, AAV3b, AAV3b.AR2.12 and AAVrh10.

In an embodiment, the AAV capsid is an AAV8 capsid. In an embodiment, the AAV capsid is an AAV1 capsid.

In addition to a promoter, an expression cassette and/or a vector may contain other appropriate transcription initiation, termination, enhancer sequences, efficient RNA processing signals such as splicing and polyadenylation (polyA) signals; TATA sequences; sequences that stabilize cytoplasmic mRNA; sequences that enhance translation efficiency (i.e., Kozak consensus sequence); introns; sequences that enhance protein stability; and when desired, sequences that enhance secretion of the encoded product. The expression cassette or vector may contain none, one or more of any of the elements described herein. Illustrative examples of suitable polyA sequences include SV40, bovine growth hormone (bGH), and TK polyA. Examples of suitable enhancers include, e.g., the CMV enhancer, the RSV enhancer, the alpha fetoprotein enhancer, the TTR minimal promoter/enhancer, LSP (TH-binding globulin promoter/alpha1-microglobulin/bikunin enhancer), amongst others.

In an embodiment, the viral vector includes a nucleic acid expression cassette comprising: a 5′ AAV inverted terminal repeat sequence (ITR), a promoter with optional enhancer, an EPO sequence, a poly A sequence, and a 3′ AAV ITR, wherein said expression cassette expresses a functional feline EPO in a host cell.

These control sequences are “operably linked” to the EPO construct sequences. As used herein, the term “operably linked” refers to both expression control sequences that are contiguous with the gene of interest and expression control sequences that act in trans or at a distance to control the gene of interest.

The expression cassette may be engineered onto a plasmid which is used for production of a viral vector. The minimal sequences required to package the expression cassette into an AAV viral particle are the AAV 5′ and 3′ ITRs, which may be of the same AAV origin as the capsid, or of a different AAV origin (to produce an AAV pseudotype). In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, an expression cassette for an AAV vector comprises an AAV 5′ ITR, the propeptide-EPO active peptide coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used.

The abbreviation “sc” refers to self-complementary. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717: and 7,456,683, each of which is incorporated herein by reference in its entirety.

An adeno-associated virus (AAV) viral vector is an AAV Dnase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. AAV serotypes may be selected as sources for capsids of AAV viral vectors (Dnase resistant viral particles) including, e.g., AAV1, AAV2, AAV3, AAV4, AAV5, AAV6, AAV6.2, AAV7, AAV8, AAV9, rh10, AAVrh64R1, AAVrh64R2, rh8, rh.10, variants of any of the known or mentioned AAVs or AAVs yet to be discovered. In one embodiment, the AAV is an AAV8 capsid, or a variant thereof. In another embodiment, the AAV is an AAV1 capsid, or a variant thereof. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). Alternatively, a recombinant AAV based upon any of the recited AAVs, may be used as a source for the AAV capsid. These documents also describe other AAV which may be selected for generating AAV and are incorporated by reference. In some embodiments, an AAV cap for use in the viral vector can be generated by mutagenesis (i.e., by insertions, deletions, or substitutions) of one of the aforementioned AAV Caps or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned Caps. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV1 capsid. In one embodiment, the AAV capsid shares at least 95% identity with an AAV8 capsid. When determining the percent identity of an AAV capsid, the comparison may be made over any of the variable proteins (e.g., vp1, vp2, or vp3). In one embodiment, the AAV capsid shares at least 95% identity with the AAV8 vp3. In another embodiment, the AAV capsid shares at least 95% identity with an AAV1 vp3. In another embodiment, a self-complementary AAV is used.

For packaging an expression cassette into virions, the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. Additionally or alternatively, a chimeric AAV capsid may be utilized. Still other AAV components may be selected. Sources of such AAV sequences are described herein and may also be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank1®, PubMed®, or the like.

Methods for generating and isolating AAV viral vectors suitable for delivery to a subject are known in the art (see, e.g., U.S. Pat. No. 7,790,449: U.S. Pat. No. 7,282,199; WO 2003/042397; WO 2005/033321, WO 2006/110689; U.S. Pat. No. 7,588,772 B2 and WO 2017/040524, the contents of which are incorporated herein by reference in their entirety). In an embodiment, a producer cell line is transiently transfected with a construct that encodes the transgene flanked by inverted terminal repeats (ITR) and a construct(s) that encodes rep and cap. In another embodiment, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally. e.g., Zhang et al. (2009, Human Gene Therapy 20:922-929, the entire contents of which are incorporated herein by reference). Methods of making and using these and other AAV production systems are also described in the following U.S. patents, the entire contents of which are incorporated herein by reference: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally. e.g., Grieger & Samulski, 2005, Adv. Biochem. Engin/Biotechnol. 99: 119-145; Buning et al., 2008, J. Gene Med. 10:717-733). Suitable methods for constructing any of the embodiments described herein will be familiar to persons skilled in the art of nucleic acid manipulation, illustrative examples of winch include genetic engineering, recombinant engineering, and synthetic techniques (see, e.g., Green and Sambrook et al., Molecular Cloning: A Laboratory Manual, Cold Spring Harbor Press, Cold Spring Harbor, NY (2012)). Similarly, methods of generating rAAV virions will be well known to persons skilled in the art (see, e.g., K. Fisher et al, (1993) J. Virol., 70:520-532 and U.S. Pat. No. 5,478,745).

Also provided herein are compositions that include the viral vector constructs described herein. The pharmaceutical compositions described herein are designed for delivery to subjects in need thereof by any suitable route or a combination of different routes. Direct delivery to the liver (optionally via intravenous, via the hepatic artery, or by transplant), oral, inhalation, intranasal, intratracheal, intraarterial, intraocular, intravenous, intramuscular, subcutaneous, intradermal, and other parental routes of administration. The viral vectors described herein may be delivered in a single composition or multiple compositions. Optionally, two or more different AAV may be delivered, or multiple viruses (see, e.g., WO 2011/126808 and WO 2013/049493). In another embodiment, multiple viruses may contain different replication-defective viruses (e.g., AAV and adenovirus).

The replication-defective viruses can be formulated with a physiologically acceptable carrier for use in gene transfer and gene therapy applications. In the case of AAV viral vectors, quantification of the genome copies (“GC”) may be used as the measure of the dose contained in the formulation or suspension. Any method known in the art can be used to determine the genome copy (GC) number of the replication-defective virus compositions of the invention. One method for performing AAV GC number titration is as follows: Purified AAV vector samples are first treated with Dnase to eliminate un-encapsidated AAV genome DNA or contaminating plasmid DNA from the production process. The Dnase resistant particles are then subjected to heat treatment to release the genome from the capsid. The released genomes are then quantitated by real-time PCR using primer/probe sets targeting specific region of the viral genome (usually poly A signal).

### Doses and Dosage Forms

As noted elsewhere herein, the present invention is predicated, at least in part, on the inventors' surprising finding that (i) a dose of an adenovirus vector that is adapted to drive the expression of recombinant feline EPO and is otherwise sufficient to increase the haematocrit or packed cell volume in a healthy feline subject, is unexpectedly detrimental or fatal to a feline subject with anaemia; (ii) that red blood cell homeostasis can nevertheless be restored in an anaemic feline subject when the feline EPO-expressing adenovirus vector is administered to the anaemic feline subject at a lower dose that would not be expected to increase the haematocrit or packed cell volume in a healthy feline subject and (iii) the low dose treatment regimen described herein minimizes the risk of polycythemia in the anaemic animals.

The dose effective to promote or otherwise restore red blood cell homeostasis in a feline subject, in particular an anaemic feline subject, is preferably about 2×10⁹ gc/kg or less. In an embodiment, the dose effective to promote or otherwise restore red blood cell homeostasis in a feline subject, in particular an anaemic feline subject, is preferably about 1×10⁹ gc/kg or less. It is to be understood that the lower limit of the dose range can be any number less than about 2×10⁹ gc/kg, other than zero, that is effective to promote or otherwise restore red blood cell homeostasis in a feline subject, preferable to promote or otherwise restore red blood cell homeostasis in the feline subject by about 70 days following commencement of treatment. In an embodiment, the dose is from about 1×10⁸ to 2×10⁹ gc/kg. In an embodiment, the dose is from about 1×10⁸ to 1×10⁹ gc/kg. In an embodiment, the dose is from about 2×10⁸ to 1×10⁹ gc/kg. In an embodiment, the dose is from about 2×10⁸ to 6×10⁸ gc/kg. In an embodiment, the dose is from about 5.5×10⁸ to about 1.4×10⁹ gc/kg. In an embodiment, the dose is from about 2×10⁸ to 1.8×10⁹ gc/kg.

In an embodiment, the dose is (i) about 3.65×10⁹ genome copies for a feline subject weighing from 2 to 6 kg or (ii) about 7.30×10⁹ genome copies for a feline subject weighing more than 6 kg. In an embodiment, the dose is about 7.30×10⁹ genome copies for a feline subject weighing more than 6 kg.

Where expression of the feline EPO is driven by a weak promoter, as described elsewhere herein, the dose may be adjusted accordingly to achieve the same or substantially the same level of expression as compared to a strong promoter. In an embodiment, where a weak promoter is employed, the dose effective to promote or otherwise restore red blood cell homeostasis in the feline subject is a dose sufficient to achieve a level of expression of feline EPO in the feline subject that is equivalent to the level of expression achieved by a dose of about 3×10⁹ gc/kg or less when a strong promoter is employed. A dose sufficient to achieve a level of expression of feline EPO in the feline subject that is equivalent to the level of expression achieved by a dose of about 2×10⁹ gc/kg or less or, alternatively, by a dose of about 1×10⁹ gc/kg or less, when a strong promoter is employed can be determined by persons skilled in the art. Such doses may suitably be achieved by administering an amount greater than about 1×10⁹ gc/kg. Alternatively, or in addition, such doses may suitably be achieved by consecutive doses; that is, more than one (e.g., 2, 3, 4, 5, 6, 7, 8, 9 and so on) consecutive doses.

In some embodiments, the amount of viral genome may be delivered in split doses. In an embodiment, the constructs may be delivered in volumes from 1 μL to about 100 mL for a veterinary subject (see e.g., Diehl et al, 2001, J. Applied Toxicology, 21:15-23 for a discussion of good practices for administration of substances to various veterinary animals, the contents of which are incorporated herein by reference in their entirety). Thus, as used herein, the term “dosage” or “dose” can refer to the total dosage delivered to the subject in the course of treatment, or the amount delivered in a single (of multiple) administration. In an embodiment, the dose administered to the feline subject is from about 1.0×10⁹ to about 5.0×10⁹ genome copies. In an embodiment, the dose administered to the feline subject is from about 1.2×10⁹ to about 5.0×10⁹ genome copies. In an embodiment, the dose administered to the feline subject is (i) about 3.65×10⁹ genome copies in a feline subject weighing from 2 to 6 kg or (ii) about 7.30×10⁹ genome copies in a feline subject weighing more than 6 kg. In an embodiment, two doses are administered to a feline subject weighing more than 6 kg, each dose comprising about 3.65×10⁹ genome copies.

Suitable methods fort delivering the recombinant vectors to host cells will be known to persons skilled in the art. The rAAV, preferably suspended in a physiologically compatible carrier, diluent, excipient and/or adjuvant, may be administered to a feline subject. Suitable carriers may be readily selected by persons skilled in the art in view of the indication for which the transfer virus is directed. For example, a suitable carrier includes saline, which may be formulated with a variety of buffering solutions (e.g., phosphate buffered saline). Other illustrative examples of suitable carriers include sterile saline, lactose, sucrose, calcium phosphate, gelatin, dextran, agar, pectin, peanut oil, sesame oil, and water.

Optionally, the compositions described herein may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The viral vectors and other constructs described herein may be used in preparing a medicament for delivering an EPO construct to a feline subject in need thereof and/or for treating chronic kidney disease in a feline subject. Thus, in another aspect, a method of treating chronic kidney disease in a feline subject is provided. Thus, in an embodiment, the feline subject has chronic kidney disease. In another embodiment, the composition is administered intravenously to the feline subject.

In an embodiment, the method includes administering a composition as described herein to a subject in need thereof. In one embodiment, the composition includes a viral vector containing an EPO expression cassette, as described herein. In one embodiment, the subject is a mammal. In another embodiment, the subject is a feline. In yet another aspect a method of treating anemia is provided. The method includes administering a composition as described herein to a subject in need thereof. In an embodiment, the composition includes a viral vector containing an EPO expression cassette, as described herein. In one embodiment, the subject is a mammal. In another embodiment, the subject is a feline.

In an embodiment, a method for treating chronic kidney disease in a feline is provided. The method includes administering an AAV viral vector comprising a nucleic acid molecule comprising a sequence encoding feline EPO.

In another aspect disclosed herein, there is provided a method of treating an anaemic feline subject with chronic kidney disease, the method comprising administering to the subject a composition comprising a recombinant adeno-associated virus (rAAV), as described herein, wherein the composition is administered to the subject at a dose of about 2×10⁹ genome copies per kg body weight or less. In another aspect disclosed herein, there is provided use of a recombinant adeno-associated virus (rAAV) as described herein, wherein the composition is formulated for administration at a dose of about 2×10⁹ genome copies per kg body weight or less. In another aspect disclosed herein, there is provided a pharmaceutical composition for use in treating an anaemic feline subject with chronic kidney disease, wherein the composition comprises a recombinant adeno-associated virus (rAAV), as described herein, wherein the composition is formulated for administration at a dose of about 2×10⁹ genome copies per kg body weight or less.

A course of treatment may optionally involve repeat administration of the same viral vector (e.g., an AAV8 vector or an AAV1 vector) or a different viral vector (e.g., an AAV8 and an AAVrh10; an AAV1 and an AAV8; an AAV1 and an AAVrh10; and so on). Other combinations may also be selected using suitable viral vectors, including those described herein. The composition described herein may also be suitably combined in a regimen involving, for example, other drugs or protein-based therapies, including e.g., recombinant feline EPO protein, and/or lifestyle changes such as dietary and exercise regimens.

The term “regulation” or variations thereof as used herein refers to the ability of a composition to inhibit one or more components of a biological pathway.

In another aspect disclosed herein, there is provided a unit dosage form comprising the recombinant adeno-associated virus (rAAV) described herein, wherein the dosage form comprises the rAAV in an amount of from about 1.0×10⁹ to about 5.0×10⁹ genome copies. In an embodiment, the unit dosage form comprises the rAAV in an amount of from about 1.2×10⁹ to about 5.0×10⁹ genome copies. In an embodiment, the unit dosage form comprises the rAAV in an amount of about 1.2×10⁹ genome copies. In an embodiment, the unit dosage form comprises the rAAV in an amount of about 2.4×10⁹ genome copies. In an embodiment, the unit dosage form comprises the rAAV in an amount of about 3.65×10⁹ genome copies. In an embodiment, the unit dosage form comprises the rAAV in an amount of about 4.86×10⁹ genome copies. In an embodiment, the dosage form is an intramuscular dosage form.

The present disclosure also extends to a kit comprising the composition or dosage form described herein. The kit may further comprise instructions, including for using the composition or dosage form in accordance with the methods and uses described herein. The kit may further comprise the composition or dosage form described herein in a lyophilised form. Suitable methods for preparing the lyophilised form will be familiar to persons skilled in the art. Where the kit comprises the composition or dosage form in a lyophilised form, the kit may further comprise a separate container comprising a pharmaceutically acceptable diluent for reconstituting the lyophilised form prior to administration, including for intramuscular administration in accordance with the methods and uses described herein.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

### Example 1—Construction of AAV-Feline EPO Expression Vector

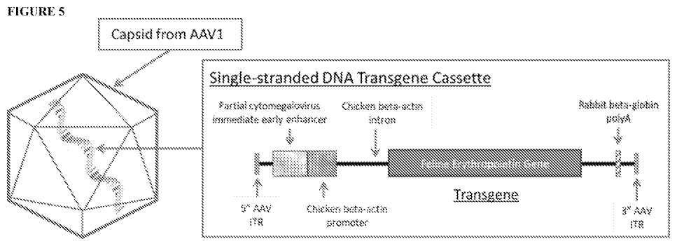

The nucleic acid sequence encoding a feline EPO proprotein was produced by codon-optimizing the nucleic acid sequence encoding the feline EPO of GenBank Accession No. AFN85670.1; SEQ ID NO:1). The codon-optimized nucleic acid sequence (SEQ ID NO:2) was cloned into an expression vector containing a chicken beta-actin promoter with a partial CMV immediate early enhancer. The expression construct was flanked by two (5′ and 3′) AAV2 inverted terminal repeats (ITR). The feline construct was packaged in an AAV serotype 1 (AAV1) capsid following triple transfection with helper and trans plasmids. It was then purified via iodixanol gradient purification and titred by Taqman quantitative PCR. The AAV1-feline EPO expression vector is shown in FIG. 5 and also referred to herein as SB-001.

### Example 2—AAV-Mediated Expression of Feline Erythropoietin in Normal Cats

To evaluate the safety and haematological responses to the administration of the AAV1-feline EPO expression vector, SB-001, a single intramuscular (IM) dose was administered to healthy, adult cats at various doses.

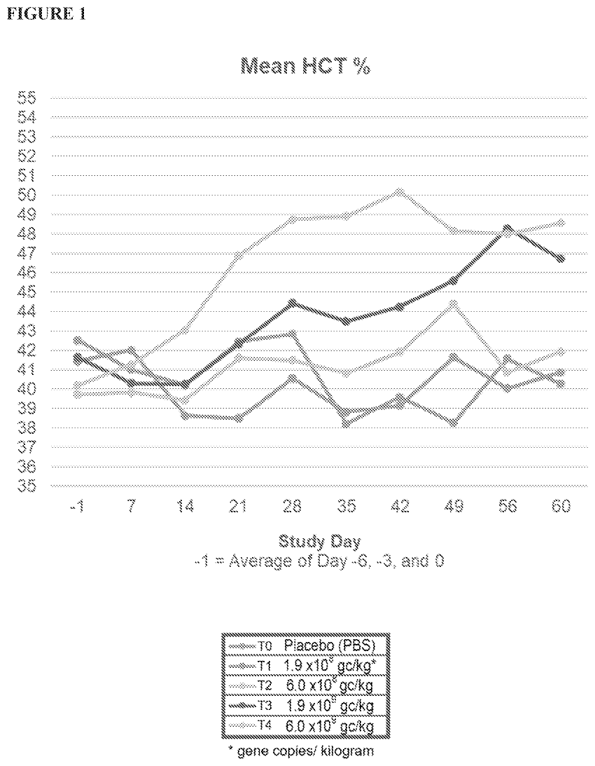

This investigation was undertaken as a single-site, non-clinical laboratory study with a randomized, controlled, parallel design. Thirty-four (34) cats were acclimated to study conditions for 7 days before 30 cats were selected to enter the dosing phase. Cats were randomly allocated to five sex-balanced groups before receiving a single IM dose of SB-001 or phosphate buffered saline placebo. Dose groups were treated once on Day 0 as shown in FIG. 1. Injection site evaluations, physical examinations, and body weights were assessed at various time points pre- and post-dosing. Blood was collected for clinical chemistry and potential immunogenicity assessments at time points pre- and post-dosing. Clinical observations to assess overall health were conducted daily. Comprehensive haematology evaluations using an automated analyzer occurred three times prior to dosing, then weekly throughout the study period. In addition, manual PCV/TP measurements were conducted once weekly to monitor cats with excessively high HCT levels, as needed. On Days 56 and 60 post-dosing, manual PCV/TP data were generated for all cats. At the end of the study, all cats dosed with SB-001 were euthanized and necropsied.

Following a single IM injection, SB-001 produced sustained and statistically significant changes in HCT at the two highest dose levels evaluated (T3; 1.9×10⁹ gene copy per kilogram body weight (gc/kg) and T4; 6.0×10⁹ gc/kg). The lowest doses (T1; 1.9×10⁸ gc/kg and T2; 6.0×10⁸ gc/kg) did not produce any consistent changes in HCT over the course of the study. The observed changes to the erythrogram in groups T2, T3, and T4 were largely anticipated and none of these findings were considered additional clinically significant safety signals.

A single dose of SB-001 given by intramuscular injection produced sustained and statistically significant changes in HCT at a dose of 1.9×10⁹ (T3) and 6.0×10⁹ gc/kg (T4) in healthy cats. This red cell haematological response was sustained over a period of 62/63 days, with no clinically significant safety concerns being identified at any dose level (FIG. 1). These data are largely consistent with previous studies reported by Walker et al. (2005, AJVR, 66(3):450-456) and Beall et al. (2000, Gene Therapy, 7:534-539). For instance, Beall et al. showed that only a dose of 1.25×10¹² gc/kg of an adenovirus-feline EPO expression vector was effective at increasing the HCT of healthy feline subjects. The lowest dose of 1.25×10¹⁰ gc/kg was shown to be ineffective. Similarly, Walker et al. showed that, of the doses tested (about 1.5×10⁷, 1.5×10⁸ and 1.5×10⁹ gc/kg), only the highest dose (1.5×10¹⁰ gc/kg) showed an increase in HCT in healthy cats, whereas there was no effect at any of the lower doses tested.

In light of the aforementioned data from healthy cats, the inventors took an average dose of 3.0×10⁹ gc/kg to investigate the effect of the SB-001 construct in anaemic cats with chronic kidney disease. This is set out in Example 3, below.

### Example 3—Dosage Study of AAV-Mediated Expression of Feline Erythropoietin in Cats with Chronic Kidney Disease

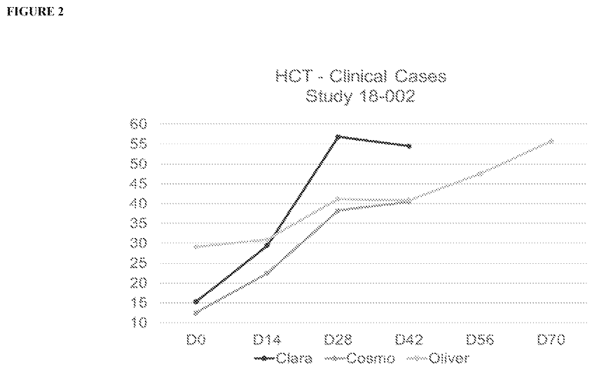

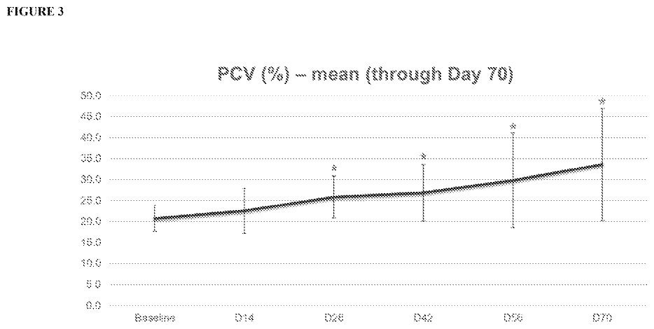

In this study, three cats (Clara, Cosmo and Oliver), each diagnosed with anaemia associated with chronic kidney disease (CKD), received SB-001 (IM; 3.0×10⁹ gc/kg). Surprisingly, the 3.0×10⁹ gc/kg dose of SB-001, which was otherwise found to be effective at increasing HCT in healthy cats, was not well tolerated or was fatal in the anaemic cats. These results are shown in FIG. 2 and further described below:
Oliver: 18.5 year old, MC Maine Coon
   Polycythemia due to SB-001 EPO at Day 70 (PCV 29.1% to 55.7%=increase of 26.6 points)
   Day 89—died with acute necrotizing pancreatitis
Clara 19.3 year old, FS, DSH
   Polycythemia at day 28 due to SB-001 EPO (PCV 15.3% to 54.5%=increase of 39.2 points)
   Seizure at D27, 2 phlebotomies
   Euthanized at D55 due to CKD progression
Cosmo: 17 year old, MC, DSH
   IRIS IV: Creatinine/BUN at enrollment:
   PCV was increasing too rapidly, if had been able to continue on study would likely have developed polycythemia (PCV 12% to 40.9%=increase of 29 points)
   Euthanized at D46—progression of CKD

### Example 4—Optimisation of AAV-Mediated Expression of Feline Erythropoietin in Cats with Chronic Kidney Disease

This study sought to investigate whether lower doses of SB-001 may be effective in anaemic cats, despite the earlier findings that lower doses are ineffective at promoting red blood cell homeostasis in healthy cats (as noted in Example 2, above, and consistent with the findings by Beall et al. and Walker et al.). This study design was similar to the study of Example 3, above, with the exception it was not blinded and no placebo was used. Animals were administered a dose of SB-001 from about 2.0×10⁸ to 6.0×10⁸ gc/kg.

Following the enrollment of the first 7 cats, an amendment was made to allow cats not responding to the therapy to receive a 3× boosted dose at Day 42. Seven of the eight boosted-dose cats met success criteria (increase of PCV into the normal range or increase of at least 5 PCV percentage points). Without treatment, a cat's PCV would be expected to decrease over time.

Efficacy was shown by either stabilizing or increasing the PCV (measured from whole blood spun down using microhaematocrit) following treatment.

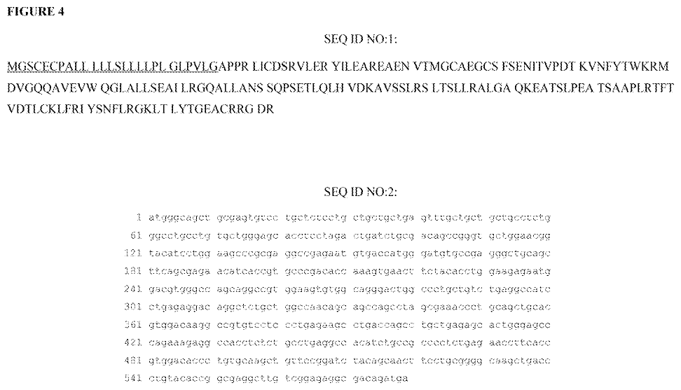

Surprisingly, a mean increase in PCV from 22.5% to 33.6% was observed in the study cohort (FIG. 3) and no cats required treatment for polycythemia. These results were surprising and unexpected, noting that similar doses of SB-001 were ineffective at increasing HCT or PCV is healthy cats.

### Example 5—Dose Optimization of AAV-Mediated Expression of Feline Erythropoietin in Cats with Chronic Kidney Disease

This study sought to confirm that lower effective doses of SB-001 are effective in anaemic cats.

This study design was similar to the study of Example 4, above. Briefly, animals were administered a single dose of about 3.65×10⁹ gene copies (gc) of SB-001 at commencement of the study. This dose was equivalent to about 5.5×10⁸ to 1.4×10⁹ gc/kg body weight (BW), as shown in Table 1 below.

TABLE 1
Dose of SB-001 administered to cats

| Cat ID # | Body Weight (kg) | Dose (gc/kg BW) |
|---|---|---|
| ANP-01 | 2.6 | 1.40E+09 |
| HLN-06 | 3.1 | 1.18E+09 |
| ANP-02 | 3.7 | 9.86E+08 |
| MEU-13 | 4.1 | 8.90E+08 |
| MEU-12 | 4.2 | 8.69E+08 |
| JDF-06 | 4.3 | 8.49E+08 |
| JDF-07 | 4.4 | 8.30E+08 |
| MEU-11 | 6.7 | 5.45E+08 |

Seven of the eight treated cats met the success criteria, taken as an increase of PCV into the normal range or an increase of at least 5 PCV percentage points. Without treatment, PCV values would be expected to decrease over time.

Efficacy was shown by either stabilizing or increasing the PCV (measured from whole blood spun down using microhaematocrit) following treatment.

Surprisingly, a mean increase of PCV from 21.25% to 39.75% was observed in the study cohort, as shown in Table 2 below, and no cats required subsequent treatment for polycythemia. These results were surprising and unexpected, noting that similar doses of SB-001 were ineffective at increasing HCT or PCV in healthy cats.

TABLE 2
Changes in PCV values in cats with chronic
kidney disease treated with SB-001

| | | | | | | | p- |
|---|---|---|---|---|---|---|---|
| Visit | | n | Mean (SD) | Min | Median | Max | value |
| Screen- | Value | 8 | 21.25 (1.67) | 20 | 21.0 | 25 | |
| ing | | | | | | | |
| Day 14 | Value | 8 | 23.88 (4.76) | 18 | 23.5 | 32 | |
| | Change | 8 | 2.63 (5.04) | −3.00 | 2.50 | 10.00 | 0.1843 |
| | from | | | | | | |
| | Screening | | | | | | |
| Day 28 | Value | 8 | 30.25 (7.55) | 21 | 30.0 | 43 | |
| | Change | 8 | 9.00 (7.82) | 0.00 | 8.00 | 23.00 | 0.0140 |
| | from | | | | | | |
| | Screening | | | | | | |
| Day 42 | Value | 8 | 33.75 (10.71) | 21 | 29.5 | 52 | |
| | Change | 8 | 12.50 (10.93) | 1.00 | 8.00 | 32.00 | 0.0143 |
| | from | | | | | | |
| | Screening | | | | | | |
| Day 56 | Value | 6 | 35.17 (10.87) | 25 | 30.5 | 55 | |
| | Change | 6 | 13.50 (10.93) | 4.00 | 10.00 | 33.00 | 0.0292 |
| | from | | | | | | |
| | Screening | | | | | | |
| Day 70 | Value | 4 | 39.75 (14.03) | 23 | 39.5 | 57 | |
| | Change | 4 | 18.75 (13.23) | 3.00 | 18.50 | 35.00 | 0.0659 |
| | from | | | | | | |
| | Screening | | | | | | |

### Example 6—Survival of Anaemic Cats with End-Stage Chronic Kidney Disease Following Treatment with SB-001

This study sought to determine the effect of lower effective doses of SB-001 on survival in anaemic cats with end-stage (Stage 4) chronic kidney disease. This study was similar to the study set out in Examples 4 and 5, above. Briefly, anaemic cats with end-stage (Stage 4) chronic kidney disease were administered SB-001 at a total dose of from about 2×10⁸ to 1.8×10⁹ gc/kg body weight, including a second dose administered, if needed, as described in Example 4 above.

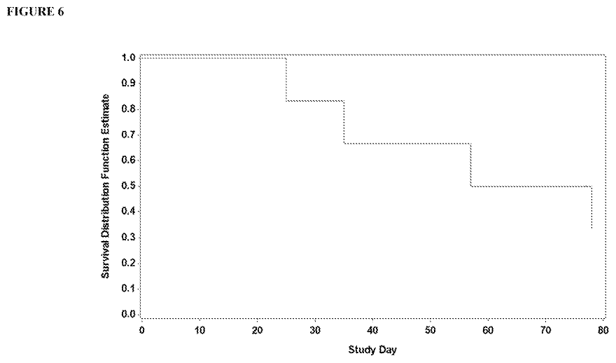

Surprisingly, 4 of 9 cats treated with low dose SB-001 lived longer than 99 days. This is to be contrasted with the findings of Boyd et al. (2008; J Vet Intern Med 2008:22:1111-1117), which showed that the survival median of cats from diagnosis of Stage 4 chronic kidney disease to death was 35 days (see Table 2 of Boyd et al. 2008). Stage 4 cats treated with SB-001 had an increased median survival time (days) of 64 days (see FIG. 6 and Table 3).

TABLE 3
Survival of anaemic cats treated with SB-001 - from
diagnosis of Stage 4 chronic kidney disease to death

| | Body Weight | Total Dose | Survival |
|---|---|---|---|
| Cat ID # | (kg) | (gc/kg body weight) | (days) |
| MEU-09 | 2.0 | 6.00E+08 | 23 |
| MEU-07 | 3.5 | 3.43E+08 | 33 |
| JDF-04 | 5.8 | 2.07E+08 | 47 |
| MEU-12 | 4.2 | 8.69E+08 | 60 |
| MEU-01 | 2.7 | 4.44E+08 | 64 |
| JDF-05 | 2.7 | 1.80E+09 | 125 |
| JDF-03 | 3.2 | 1.52E+09 | 158 |
| HLN-01 | 5.8 | 2.07E+08 | 160 |
| HLN-03 | 8.4 | 2.86E+08 | 339 |

When the survival data from the feline subjects of Examples 3-6 were collated, it was surprisingly found that cats treated with low dose SB-001 lived more than three times as long from time of treatment when compared to feline subjects that received alternative interventions, as described in Boyd et al. (2008). These findings are presented in Table 4 below.

TABLE 4
Survival median of anaemic cats with end-stage (Stage
4) chronic kidney disease - from diagnosis to death

| | Number of | Median | Lower 95% | Upper 95% |
|---|---|---|---|---|
| | Animals | (days) | CI (days) | CI (days) |
| Treatment with | n = 23 | 96 | 60 | 160 |
| low dose SB-001 * | | | | |
| Other | n = 42 | 25 | 6 | 74 |
| intervention ** | | | | |

* Survival from Day 0 (first treatment day); data from all SB-001-treated cats that participated in the studies described at Examples 3-6 above (n = 23).
** As described in Table 3 of Boyd et al. (2008)

### Example 7—Treatment of Anaemic Cats with Chronic Kidney Disease with AAV8-Mediated Expression of Feline Erythropoietin

The nucleic acid sequence encoding a feline EPO proprotein will be obtained from GenBank (e.g., Accession No. AFN85670). The sequence will be cloned into an expression vector containing a chicken beta-actin promoter with a partial CMV immediate early enhancer. The expression construct will be flanked by two (5′ and 3′) AAV2 inverted terminal repeats (ITR). The feline construct will be packaged in an AAV serotype 8 (AAV8) capsid by triple transfection and iodixanol gradient purification and titred by Taqman quantitative PCR to produce an AAV8-feline EPO expression vector.

Anaemic cats will be administered an intramuscular (IM) dose of the AAV8-feline EPO expression vector of about 2.0×10⁹ gc/kg or less and changes in HCT and/or PCV will be determined as described elsewhere herein. Efficacy will be shown by either a stabilization or increase in PCV into the normal range or an increase of at least 5 PCV percentage points (e.g., as measured from whole blood spun down using microhaematocrit).

1. A method for promoting red blood cell homeostasis in a feline subject with anaemia, the method comprising administering to a subject in need thereof a composition comprising a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the composition is administered to the subject at a dose effective to promote red blood cell homeostasis in the feline subject, wherein the dose is about 2×10⁹ genome copies per kg body weight (gc/kg) or less.

2. The method of claim 1, wherein the dose is about 1×10⁹ gc/kg or less.

3. The method of claim 1, wherein the dose is from about 1×10⁸ to 2×10⁹ gc/kg.

4. The method of claim 1, wherein the dose is from about 1×10⁸ to 1×10⁹ gc/kg.

5. The method of claim 1, wherein the dose is from about 2×10⁸ to 1×10⁹ gc/kg.

6. The method of claim 1, wherein the dose is from about 2×10⁸ to 6×10⁸ gc/kg.

7. The method of any one of claims 1 to 6, wherein the feline subject with anaemia has a packed cell volume (PCV) of about 28% or less.

8. The method of claim 7, wherein the feline subject with anaemia has a PCV in a range of about 10% to about 28%.

9. The method of claim 7, wherein the feline subject with anaemia has a PCV in a range of about 22% to about 28%.

10. The method of claim 7, wherein the feline subject with anaemia has a PCV of about 22.5%.

11. The method of any one of claims 1 to 10, wherein the dose is effective to increase the PCV in the feline subject to a value from about 30% to about 55% by day 70 following administration of the composition.

12. The method of claim 11, wherein the dose is effective to increase the PCV in the feline subject to a value from about 30% to about 35% by day 70 following administration of the composition.

13. The method of claim 11, wherein the dose is effective to increase the PCV in the feline subject to a value from about 33% to about 35% by day 70 following administration of the composition.

14. The method of any one of claims 1 to 13, wherein the dose is effective to increase the PCV in the feline subject by an amount from about 5 to about 30 PCV % points by day 70 following administration of the composition.

15. The method of claim 14, wherein the dose is effective to increase the PCV in the feline subject by an amount from about 10 to about 20 PCV % points by day 70 following administration of the composition.

16. The method of claim 14, wherein the dose is effective to increase the PCV in the feline subject by about 15 PCV % points by day 70 following administration of the composition.

17. The method of any one of claims 1 to 16, wherein the feline subject does not require treatment for polycythemia.

18. The method of any one of claims 1 to 17, wherein the composition is administered intramuscularly as a unit dose of from about 1.0×10⁹ to about 5.0×10⁹ genome copies.

19. The method of any one of claims 1 to 18, wherein the method comprises administering to the feline subject a subsequent dose of the composition.

20. The method of claim 19, wherein the subsequent dose is administered to the feline subject from about day 28 to about day 56 following initial administration of the composition.

21. The method of claim 19, wherein the subsequent dose is administered to the feline subject from about day 40 to about day 45 following initial administration of the composition.

22. The method of any one of claims 1 to 21, wherein the feline EPO is a full length feline EPO protein.

23. The method of any one of claims 1 to 22, wherein the nucleic acid sequence encodes the mature feline EPO protein in combination with a heterologous leader sequence.

24. The method of any one of claims 1 to 23, wherein the feline EPO sequence comprises the amino acid sequence of SEQ ID NO:1 or an amino acid sequence having at least 70% sequence identity thereto.

25. The method of any one of claims 1 to 24, wherein the nucleic acid sequence encoding the feline EPO comprises the nucleic acid sequence of SEQ ID NO:2 or a nucleic acid sequence having at least 70% sequence identity thereto.

26. The method of claim 25, wherein the nucleic acid sequence is codon optimized.

27. The method of any one of claims 1 to 26, wherein the expression control sequence comprises a promoter.

28. The method of claim 27, wherein the promoter is a CB7 promoter.

29. The method of claim 27, wherein the promoter is a TBG promoter.

30. The method of claim 27, wherein the expression control sequence comprise a tissue-specific promoter.

31. The method of claim 30, wherein the tissue-specific promoter is a kidney-specific promoter or a muscle-specific promoter.

32. The method of claim 30, wherein the tissue-specific promoter is selected from a Nkcc2 promoter, uromodulin promoter, Ksp-cadherin promoter and THP gene promoter.

33. The method of any one of claims 1 to 32, wherein the AAV capsid further comprises one or more of an intron, a Kozak sequence, a polyA, and post-transcriptional regulatory elements.

34. The method of any one of claims 1 to 33, wherein the AAV capsid is selected from the group consisting of AAV1, AAV5, AAV6, AAV8, AAVrh64R1, AAV9, AAVrh91, AAVhu.37, AAV3b, AAV3b.AR2.12 and AAVrh10.

35. The method of claim 34, wherein the AAV capsid is an AAV8 capsid.

36. The method of claim 34, wherein the AAV capsid is an AAV1 capsid.

37. The method of any one of claims 1 to 36, wherein the feline subject has chronic kidney disease.

38. The method of any one of claims 1 to 37, wherein the composition is administered intravenously to the feline subject.

39. Use of a recombinant adeno-associated virus (rAAV) comprising an AAV capsid in the manufacture of a medicament for promoting red blood cell homeostasis in a feline subject with anaemia, wherein the AAV capsid comprises a nucleic acid sequence encoding feline erythropoietin (EPO) and an expression control sequence that directs expression of the EPO in the subject, wherein the rAAV is formulated for administration at a dose effective to promote red blood cell homeostasis in the feline subject, wherein the dose is about 2×10⁹ genome copies per kg body weight (gc/kg) or less.

40. The use of claim 39, wherein the composition is formulated for intramuscular administration as a unit dose of from about 1.0×10⁹ to about 5.0×10⁹ genome copies.

41. A pharmaceutical composition for use in promoting red blood cell homeostasis in a feline subject with anaemia, wherein the composition comprises a recombinant adeno-associated virus (rAAV) comprising an AAV capsid, wherein the AAV capsid comprises a nucleic acid sequence encoding feline erythropoietin (EPO) and an expression control sequence that directs expression of the EPO in the subject, wherein the rAAV is formulated for administration at a dose effective to promote red blood cell homeostasis in the feline subject, wherein the dose is about 2×10⁹ genome copies per kg body weight (gc/kg) or less.

42. The composition for use of claim 41, wherein the composition is formulated for intramuscular administration as a unit dose of from about 1.0×10⁹ to about 5.0×10⁹ genome copies.

43. A method of treating an anaemic feline subject with chronic kidney disease, the method comprising administering to the subject a composition comprising a recombinant adeno-associated virus (rAAV) comprising an AAV1 capsid, wherein the AAV1 capsid comprises a nucleic acid sequence encoding feline erythropoietin (EPO) having an amino acid sequence of SEQ ID NO:1, and an expression control sequence that directs expression of the EPO in the subject, wherein the composition is administered intramuscularly at a dose of (i) about 3.65×10⁹ genome copies to a feline subject weighing from 2 to 6 kg or (ii) about 7.30×10⁹ genome copies to a feline subject weighing more than 6 kg.

44. The method of claim 43, wherein the rAAV is administered at a dose of about 7.30×10⁹ genome copies, administered in the form of two doses each comprising about 3.65×10⁹ genome copies, to a feline subject weighing more than 6 kg.

45. Use of a recombinant adeno-associated virus (rAAV) comprising an AAV1 capsid in the manufacture of a medicament for treating an anaemic feline subject with chronic kidney disease, wherein the AAV1 capsid comprises a nucleic acid sequence encoding a feline erythropoietin (EPO) having an amino acid sequence of SEQ ID NO:1, and an expression control sequence that directs expression of the EPO in the subject, wherein the rAAV is formulated for intramuscular administration at a dose of (i) about 3.65×10⁹ genome copies to a feline subject weighing from 2 to 6 kg or (ii) about 7.30×10⁹ genome copies to a feline subject weighing more than 6 kg.

46. The use of claim 45, wherein the rAAV is formulated for intramuscular administration at a dose of about 7.30×10⁹ genome copies, administered in the form of two doses each comprising about 3.65×10⁹ genome copies, to a feline subject weighing more than 6 kg.

47. A pharmaceutical composition for use in treating an anaemic feline subject with chronic kidney disease, wherein the composition comprises a recombinant adeno-associated virus (rAAV) comprising an AAV1 capsid, wherein the AAV1 capsid comprises a nucleic acid sequence encoding a feline erythropoietin (EPO) having an amino acid sequence of SEQ ID NO:1, and an expression control sequence that directs expression of the EPO in the subject, wherein the rAAV is formulated for intramuscular administration at a dose of (i) about 3.65×10⁹ genome copies to a feline subject weighing from 2 to 6 kg or (ii) about 7.30×10⁹ genome copies to a feline subject weighing more than 6 kg.

48. The composition for use of claim 47, wherein the rAAV is formulated for intramuscular administration at a dose of about 7.30×10⁹ genome copies, administered in the form of two doses each comprising about 3.65×10⁹ genome copies, to a feline subject weighing more than 6 kg.

49. A unit dosage form comprising a recombinant adeno-associated virus (rAAV) comprising an AAV capsid, wherein the AAV capsid comprises a nucleic acid sequence encoding feline erythropoietin (EPO) and an expression control sequence that directs expression of the EPO in a feline subject, wherein the unit dosage form comprises the rAAV in an amount of from about 1.2×10⁹ to about 5.0×10⁹ genome copies.

50. The unit dosage form of claim 49, wherein the unit dosage form comprises the rAAV in an amount of about 3.65×10⁹ genome copies.

51. The unit dosage form of claim 49 or claim 50, wherein the dosage form is an intramuscular dosage form.

52. A kit comprising the unit dosage form of any one of claims 49 to 51.
